SEQUENCE LISTING (1 sequence)

SEQ ID NO: 1 - protein, 183 aa, Hepatitis B Virus
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRGRSPRRRTPSPRRRRSQSPRRRRSQSREPQC

(19) United States
(12) Patent Application Publication
(10) Pub. No.: US 2022/0356538 A1
(43) Pub. Date: Nov. 10, 2022

(54) HEPATITIS B VIRUS MUTANTS WITH INCREASED COVALENTLY CLOSED CIRCULAR DNA

(71) Applicant: The Penn State Research Foundation, University Park, PA (US)

(72) Inventors: Jianming HU, Hummelstown, PA (US); Ji XI, Hershey, PA (US)

(21) Appl. No.: 17/763,109

(22) PCT Filed: Sep. 23, 2020
(86) PCT No.: PCT/US2020/052186

§ 371 Date: Mar. 23, 2022

Related U.S. Application Data
(60) Provisional application No. 62/904,727, filed on Sep. 24, 2019.

(51) Int. Cl. C12Q 1/70; C12N 15/11; C12N 15/63

(52) U.S. Cl. CPC C12Q 1/706; C12N 15/11; C12N 15/63

(57) ABSTRACT

Provided are compositions and methods for identifying test agents as candidate for use in reducing hepatitis B virus (HBV). The methods involve testing one or more test agents against a mutated HBV core (HBc), wherein the HBc mutation is at HBc amino acid position HBc protein amino acid position 28, 30, 82, 84, 98, 100, 141, 143, 145, 146, 147, 148 or 149 of SEQ ID NO:1 of the HBc. Amounts of HBV cccDNA produced by HBV containing the mutated HBc are determined. The method can be performed in vitro or in vivo. A reduction in cccDNA relative to a control indicates the test agent in the test container is a candidate for reducing HBV in an individual. Cell cultures divided into reaction containers that each contain a distinct test agent are also included. Modified non-human mammals that express or include the mutant HBc proteins are included.

### CROSS REFERENCE TO RELATED APPLICATIONS

This application claims priority to U.S. provisional patent application 62/904,727, filed Sep. 24, 2019, the entire disclosure of which is incorporated herein by reference.

### STATEMENT REGARDING FEDERALLY SPONSORED RESEARCH

This invention was made with government support under contract no. R01A1043453 awarded by the National Institutes of Health. The government has certain rights in the invention.

### FIELD

The present invention relates generally to Hepatitis B Virus (HBV) infection and more particularly to identifying agents that can target HBV covalently closed circular (CCC) DNA using mammalian cells in culture, and using animal models.

### BACKGROUND

Hepatitis B virus (HBV) is a global pathogen, with hundreds of millions chronically infected worldwide who are at greatly increased risk of developing end-stage liver diseases including cirrhosis and hepatocellular carcinoma (1). Current therapies for chronic HBV infection fail to clear the virus due to the persistence of the viral genome in the host cell nucleus, an episomal DNA called the covalently closed circular DNA (cccDNA) (2).

The HBV cccDNA is derived from the viral relaxed circular DNA (rcDNA) that is contained in viral nucleocapsids (NCs) within the extracellular complete virion or in the host cell cytoplasm (1, 3). rcDNA is, in turn, synthesized by reverse transcription from a viral RNA copy of the DNA genome, the so-called pregenomic RNA (pgRNA) by the viral reverse transcriptase (RT). The HBV capsid or core (HBc) protein is a small (183 amino acid residues long) protein that forms the shell of the NC and actively participates in multiple steps of HBV replication (4, 5). HBc consists of an N-terminal domain (NTD) (from residue 1-140) and a C-terminal domain (CTD) (150-183), which are connected together by a short linker peptide (141-149). Whereas the NTD is essential for capsid assembly, it also plays a role in pgRNA packaging and reverse transcription. Similarly, the CTD is critical for pgRNA packaging and reverse transcription while also facilitates capsid assembly. Furthermore, the short linker also plays a critical role in multiple steps of HBV replication (6).

The human pathogen hepatitis B virus (HBV) belongs to the family of Hepadnaviridae, a group of small, hepatotropic DNA viruses that also include closely related animal viruses, such as the duck hepatitis B virus (DHBV). Hepadnaviruses contain a small (ca. 3-kb), partially double-stranded (ds-), relaxed circular (rc) DNA genome enclosed within an icosahedral capsid that is, in turn, formed by multiple copies (240 or 180) of the viral capsid or core protein. All hepadnaviruses replicate their genomic DNA via an RNA intermediate, termed the pregenomic RNA (pgRNA), by reverse transcription. Upon entering the host cells, the virion rcDNA is released into the nucleus for conversion into a covalently closed circular (ccc) DNA, which then serves as the viral transcriptional template for the synthesis of all viral RNAs, including pgRNA, by the host RNA polymerase II. After being packaged together with the viral reverse transcriptase (RT) protein into assembling immature nucleocapsid (NC), the pgRNA is converted by the multifunctional RT, first to a single-stranded DNA (ssDNA) and then to the characteristic rcDNA. The mature (i.e., rcDNA-containing) NCs are then encapsulated by the viral envelop proteins and secreted extracellularly as virions, or they can deliver their rcDNA content to the nucleus to be converted to more cccDNA via an intracellular cccDNA amplification pathway. Although cccDNA is clearly critical to HBV persistent infection, it is difficult to detect and quantify accurately since it exists in the infected cells only at very low levels in the presence of a large excess of structurally similar rcDNA. Methods such as PCR often required to detect the low levels of cccDNA can't readily differentiate cccDNA from rcDNA. There is thus an ongoing need for modified HBV viruses that can be used for improved methods of identifying agents that may be useful in combatting HBV infection, particularly those that target cccDNA. The present disclosure is pertinent to this need.

The HBV core protein (HBc) consists of two separate domains: the N-terminal domain (NTD), which is sufficient to form the capsid shell, and the C-terminal domain (CTD), which is not absolutely required for capsid assembly but nevertheless essential for viral replication. The CTD is highly basic and dynamically phosphorylated, which is thought to be important for viral RNA packaging and DNA synthesis. The NTD has also been shown to play a role in viral DNA synthesis beyond its role in capsid assembly.

The two alternative fates of mature NCs (i.e., envelopment versus cccDNA amplification) are known to be regulated by the viral envelope proteins. Since HBc forms the NC shell, it is also likely to play a key role in these processes. On the other hand, the HBc CTD harbors the nuclear localization signal (NLS) and thus is thought to play an important role in delivering the rcDNA in mature NCs to the nucleus for cccDNA formation. Since at least partial disassembly (uncoating) of the mature NCs is required to allow rcDNA release to the host cell nucleus for cccDNA formation, NC stability or integrity likely plays a critical role in cccDNA formation. In established human hepatoma cells in culture, which have limited ability to support HBV cccDNA formation, a processed form of rcDNA called protein-free (PF) or deproteinated (dp) rcDNA, also accumulates to high levels. PF-rcDNA is derived from rcDNA, but the viral RT protein, which is used as a protein primer to initiate viral DNA synthesis and remains attached to rcDNA in mature NCs, has been removed. At least partial uncoating of the mature NCs is also thought to be required for the removal of RT from rcDNA and the generation of the PF-rcDNA. Thus, regardless of whether PF-rcDNA is a true intermediate during the conversion of RC to cccDNA, it may be a useful marker for the uncoating of mature NCs. There is an ongoing need for improved approaches that are suitable for analyzing the effect of multiple test agents on the role of HBc in coordinating the two alternative fates of mature NCs, and how or if the formation of cccDNA can be manipulated for use in prophylaxis and/or therapy of HBV. The present disclosure is pertinent to this need.

### SUMMARY OF THE INVENTION

This disclosure relates to methods for identifying test agents as candidate for use in reducing hepatitis B virus (HBV).

In one aspect a method of the disclosure generally comprises:a) introducing at least one test agent into mammalian cells, the cells comprising one or more polynucleotides that encode an HBV genome, wherein the genome comprises a segment encoding a mutated HBV core (HBc), the HBc comprising one or more changes at amino HBc acid positions 28, 30, 82, 84, 98, 100, 141, 143, 145, 146, 147, 148, or 149 of SEQ ID NO:1. The test agent is allowed to be in contact with the mammalian cells for a period of time. Subsequently, amounts of HBV cccDNA in the mammalian cells is determined. A reduction in cccDNA relative to a control indicates the test agent is a candidate for reducing HBV in an individual. As an alternative to mammalian cells, which may be in culture or within a modified non-human mammal, the disclosure also includes using isolated mutated HBc proteins in in vitro assays to analyze test agents on cccDNA formation. The description of using the mutated HBc proteins herein, when discussing mammalian cells, applies equally to such in vitro assays.

When performed in cell culture, or using isolated proteins and test agents in vitro, the method is suitable in certain approaches for concurrently screening multiple test agents, such as by dividing cell cultures/reactions into separate reaction containers and adding a distinct test agent to the separate containers. In certain implementations the control comprises a cccDNA value produced by a control mammalian cell culture comprising one or more plasmids that encode an HBV that does not comprise any of the described mutations.

The disclosure includes mammalian cell cultures that comprise the aforementioned polynucleotides, wherein the mammalian cells of the cell culture are divided into a plurality of reaction containers, and wherein the plurality of reaction containers each contains a distinct test agent. Expression vectors comprising the polynucleotides are included. Transgenic animals comprising the polynucleotides encoding the described HBc mutations are included.

The disclosure also provides a method for screening test agents that generally comprises: a) introducing into a non-human mammalian subject a polynucleotide, or by modifying a chromosome segment, so that the non-human mammal encodes an HBV genome, wherein the genome comprises a segment encoding a mutated HBV core (HBc) protein, wherein the HBc mutation is at HBc protein position 28, 30, 82, 84, 98, 100, 141, 143, 145, 146, 147, 148, or 149 of SEQ ID NO:1, or a combination thereof; b) introducing into the mammalian subject a test agent, and subsequently, c) determining amounts of HBV cccDNA in the cells of the mammal, wherein a reduction in cccDNA in the cells of the non-human mammal relative to a control indicates the test agent is a candidate for use in reducing HBV in an individual.

The disclosure also provides a method for identifying an inhibitor of covalently closed circular (CCC) DNA production, the method comprising a) contacting a test agent with a mutated HBV core (HBc) protein comprising an HBc mutation that is at HBc protein position 28, 30, 82, 84, 98, 100, 141, 143, 145, 146, 147, 148, or 149 of SEQ ID NO:1, or a combination thereof, wherein the HBc is present in mammalian cells, within a non-human mammal, or in mammalian cells in culture, or by using isolated proteins or particles containing the HBc proteins and test agents in vitro; b) allowing the test agent to be in contact with the HBc for a period of time, and subsequently, c) determining the amount of cccDNA, wherein reduction in the amount of cccDNA relative to a control identifies the test agent as an inhibitor of cccDNA production.

In embodiments, the disclosure also includes introducing directly introducing into non-human mammals HBV which comprises mutant HBc proteins, as described herein, and using such HBV infected animals to determine the effects of test agents on the HBV infection, including but not necessarily by directly or indirectly measuring an amount of cccDNA, and comparing the amount to a suitable control.

### BRIEF DESCRIPTION OF THE FIGURES

FIG. 1. Effects of HBc linker mutations on cccDNA levels and NC stability. HBV replication plasmids expressing either the wild type (wt) HBc or indicated linker mutants were transfected into human hepatoma cells (HepG2). HBV DNA associated with NCs (core DNA) (lanes 1-8), or protein-free DNA (PF-DNA) (i.e., those DNA species no longer covalently attached to the viral RT protein and mostly released from NCs) (lanes 9-12) was extracted from the transfected cells and analyzed by Southern blot analysis using a radio-labeled HBV DNA probe. The core DNA shown in lanes 5-8 was extracted following prior treatment of the cell lysate with an exogenous DNase, which removes core DNA that was not protected by the capsid (i.e., from destabilized NCs). RC, rcDNA; CCC, cccDNA; SS, single stranded DNA—a reverse transcription intermediate during the synthesis of rcDNA from pgRNA.

FIG. 2. Effects of HBc linker mutations on virion secretion. HBV replication plasmids expressing either the wildtype (wt) HBc or indicated linker mutants were transfected into human hepatoma cells (HepG2). Secreted virions as well as naked (non-enveloped) released into the culture supernatant were resolved by native agarose gel electrophoresis and detected by a radio-labeled HBV DNA probe following transfer to nitrocellulous membrane. V, virion; Ca; naked capsid.

FIG. 3. Effects of HBc linker and envelope mutations on cccDNA levels. An HBV replication plasmid expressing either the wildtype (wt) HBc, the HBc linker mutation T146A/T147A, a mutant unable to express the large envelope protein (L), or a mutant containing both the linker and envelope mutation (T146A/T147A-L) was transfected into human hepatoma cells (HepG2). HBV DNA associated with NCs (core DNA; A), or protein-free DNA (PF-DNA; B) (i.e., those DNA species no longer covalently attached to the viral RT protein and mostly released from NCs) was extracted from the transfected cells and analyzed by Southern blot analysis using a radio-labeled HBV DNA probe. RC, rcDNA; CCC, cccDNA; SS, single stranded DNA—a reverse transcription intermediate during the synthesis of rcDNA from pgRNA.

FIG. 4. HBc linker mutants increased HBV CCC DNA by 2-30 fold. Data are shown for LC (S141T/V148I/V149I), S141A, S141D, S141R, L143A, L143I, E145D, and TT146/147AA (a combination of T146A and T147A; aka T146A/T147A). Mutant 4141-149 (deletion of residues 141-149 in the HBc amino acid sequence) and L140A blocked HBV core DNA synthesis and thus no cccDNA was made, illustrating certain mutations that are not suitable for use in the described method (panel A). Panel B shows results obtained using or protein-free DNA (PF-DNA).

FIG. 5. HBc NTD mutants increased HBV CCC DNA by 2-40 fold. Three RXL motifs in HBc NTD (R28, R82, R98) targeted, either R alone to A (lanes 2-8), or both R and L in the motif changed to A (lanes 10-16), either within each individual motif or in various combinations of the motifs. R28/82A, a combination of R28A and R82A; R28/98A, a combination of R28A and R98A; R82/98A, a combination of R82A and R98A; R28/82/98A, a combination of R28A, R82A, and R98A; RL28AA, a combination of R28A and L30A, RL82AA, a combination of R82A and L84A, RL98AA, a combination of R98A and L100A; RL28/82AA, a combination of R28A, L30A, R82A, and L84A; RL28/98AA, a combination of R28A, L30A, R98A, and L100A; RL82/98AA, a combination of R82A, L84A, R98A, and L100A; and RL28/82/98AA, a combination of R28A, L30A, R82A, L84A, R98A, and

L100A.

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The present disclosure provides compositions and methods for identifying agents as candidates for use in prophylaxis and/or therapy of HBV infection. The disclosure includes the following non-limiting aspects.

Regarding cccDNA, current evidence indicates that HBc can control cccDNA formation through at least three distinct mechanisms: 1) the release of the rcDNA-containing NC outside the cell via interactions with the vial envelope proteins to form virions for secretion (thus depleting the intracellular rcDNA pool available for cccDNA formation); 2) the import of the rcDNA to the host cell nucleus where its conversion to cccDNA takes place; and 3) the disassembly of the NC to release the rcDNA content (uncoating) so it is accessible to host cell factors that convert it to cccDNA (1, 2). We previously identified mutants of HBc that show enhanced cccDNA levels and likely exerted their effects via the 2nd or 3rd of these mechanisms (e.g., see U.S. Pat. No. 9,963,751, the disclosure of which is incorporated herein by reference). The present disclosure provides additional, novel HBc mutants, with substitutions in the short linker region, which were generated by us, and have not been previously described.

In one approach, the disclosure provides a method for screening a plurality of test agents to determine if they are candidates for use in reducing HBV in an individual. In general, the method comprises analyzing test agents using any system and determining the amount of cccDNA using particular mutants described herein is performed. In embodiments, the disclosure uses mutant HBc protein that comprises a mutation of any position of the HBc protein (SEQ ID NO:1) selected from position 28, 30, 82, 84, 98, 100, 141, 143, 145, 146, 147, 148 or 149 of SEQ ID NO:1. Thus, the disclosure included mutations of the N-terminal domain (NTD) (from residue 1-140 of SEQ ID NO:1) as well as mutations of the short linker peptide (position 141-149 of SEQ ID NO:1).

In one embodiment, the method comprises screening a plurality of test agents to identify candidates for use in reducing HBV in an individual by: a) contacting a plurality of distinct test agents with mutated HBV core (HBc) protein comprising one or more mutations at HBc protein position(s) of 28, 30, 82, 84, 98, 100, 141, 143, 145, 146, 147, 148 or 149 of SEQ ID NO:1; b) allowing the test agent to be in contact with the mutant HBc protein for a period of time, and subsequently, c) determining conversion of rcDNA to cccDNA , wherein a reduction in cccDNA relative to a control indicates the test agent is a candidate for use in reducing HBV in an individual.

In one embodiment, the disclosure comprises: a) introducing a plurality of distinct test agents into separate test containers, each test container comprising mammalian cells which comprise one or more polynucleotides that encode an HBV genome, wherein the genome comprises a segment encoding a mutated HBV core (HBc) protein, wherein the HBc mutation is of at least on HBc protein amino acid position 28, 30, 82, 84, 98, 100, 141, 143, 145, 146, 147, 148 or 149 of SEQ ID NO:1, or any combination thereof; b) allowing the test agent to be in contact with the mammalian cells for a period of time, and subsequently, c) determining amounts of HBV cccDNA in the mammalian cells in each test container, wherein a reduction in cccDNA relative to a control indicates the test agent in the test container is a candidate for reducing HBV in an individual.

In another aspect the disclosure comprises a method for screening test agent comprising: a) introducing into a non-human mammalian subject a polynucleotide encoding an HBV genome, wherein the genome comprises a segment encoding at least one described mutated HBV core (HBc) protein; b) introducing into the mammalian subject a test agent, and subsequently, c) determining amounts of HBV cccDNA in the cells of the mammal, wherein a reduction in cccDNA in the cells relative to a control indicates the test agent is a candidate for use in reducing HBV in an individual. In one embodiment, the cells of the animal are liver cells.

The amino acid sequence of HBc is known in the art and the location of the mutations disclosed herein is taken in reference to it. In an embodiment wild type HBc comprises or consists of the sequence:

(SEQ ID NO: 1)
MDIDPYKEFGATVELLSFLPSDFFPSVRDLLDTASALYREALESPEHCS
PHHTALRQAILCWGELMTLATWVGNNLEDPASRDLVVNYVNTNMGLKIR
QLLWFHISCLTFGRETVLEYLVSFGVWIRTPPAYRPPNAPILSTLPETT
VVRRRGRSPRRRTPSPRRRRSQSPRRRRSQSREPQC,

wherein the amino acids that may be mutated according to this disclosure are shown enlarged and in bold.

The disclosure includes all nucleotide sequences encoding HBc each mutant alone, and every combination of the mutants. Amino acid changes in the described sequences may be presented using conventional amino acid change notation. For example, R28/98A means both R28 and R98 were changed to A, and R28/82/98A means R28, R82, and R98 were all changed to A.

The disclosure thus includes methods relating to use of polynucleotides encoding HBc with the mutation at HBc position 28 alone, the mutation at HBc position 30 alone, the mutation at HBc position 82 alone, the mutation at HBc position 84 alone, the mutation at HBc position 98 alone, the mutation at HBc position 100 alone, the mutation at HBc position 141 alone, the mutation at HBc position 143 alone, the mutation at HBc position at 145 alone, the mutation at HBc position 146 alone, the mutation at HBc position 147 alone, the mutation of HBc position 148 alone, and the mutation of HBc position 149 alone. As stated above, the disclosure includes all combinations of the described mutants. In embodiments, the disclosure includes an HBc protein comprising two mutations, such as at HBc positions 146 and 147, or two mutations at HBc positions 28 and 82. In embodiments, the disclosure includes HBc protein comprising three mutations, such as at HBc positions 28, 82 and 98. Any amino acid may be substituted for the described HBc positions of SEQ ID NO:1. In non-limiting embodiments, a non-conservative amino acid substitution is made, but conservative substitutions are not necessarily excluded from the disclosure, such as E145D. In embodiments, an Arg is not substituted with His or Lys. In embodiments, the non-conservative substitution comprises a change of one or more of the described positions with Gly, Ala, Val, Leu, or Ile. In non-limiting embodiments, the substitution comprises replacement of one or more of the described positions of SEQ ID NO:1 with Ala, Arg, Asp, Ile, Gln, Thr, or a Ser. In non-limiting embodiments, the described mutations are selected from the following, wherein the amino acid preceding the position number(s) is the un-mutated position(s), and wherein the amino acid(s) following the position number(s) is the mutated position(s): R28A, L30A, R82A, L84A, R98A, L100A, S141A, S141D, S141R, S141T, L143A, L143I, E145D, T146A, T147A, V148I, and V149I. In embodiments, combinations of two or three mutations are provided, representative examples of which include but are not necessarily limited to R28A/R82A, R28A/R98A, R82A/R98A, R28A/R82A/R98A, R28A/L30A, R82A/L84A, R98A/L100A, R28A/L30A/R82A/L84A, R28A/L30A/R98A/L100A, R82A/L84A/R98A/L100A, R28A/L30A/R82A/L84A/R98A/L100A, T146A/T147A, and S141T/V148I/V149I. Notably, the L140A mutation blocked HBV DNA synthesis and thus no cccDNA was made, illustrating that not all tested mutations are suitable for use in the described method. The same applies to a deletion of amino acids 141-149.

In another aspect the disclosure provides a transgenic non-human mammal and a method of using it for screening test agents for use in prophylaxis and/or therapy of HBV infection, where the transgenic non-human mammal is engineered to express a mutated HBc protein, wherein the HBc mutation is as described above. The method comprises administering to the non-human mammal a test agent and determining cccDNA in a sample obtained from the mammal, wherein a reduction in cccDNA indicates that test agent is a candidate for use in reducing HBV in an individual. In one embodiment, PF-rcDNA is measured instead of cccDNA. In certain aspects PF-rcDNA comprises evidence of rcDNA processing and thus serves a putative intermediate during RC to cccDNA conversion. Accordingly, in embodiments, determining PF-rcDNA comprises determining cccDNA.

In another embodiment, the disclosure includes using isolated HBV proteins or particles, such as the mutated HBc proteins described herein, in in vitro assays to analyze the effects of test agents on the amount of cccDNA.

In another embodiment, HBV infectious particles comprising the HBc mutants, or isolated HBV DNA that encodes the HBc mutants, can be introduced into non-human mammals, and such animals can be used to test the test agents.

In more detail, the HBV cccDNA is derived from the viral relaxed circular DNA (rcDNA) that is contained in viral nucleocapsids (NCs) within the extracellular complete virion or in the host cell cytoplasm (1, 3). rcDNA is, in turn, synthesized by reverse transcription from a viral RNA copy of the DNA genome, the so-called pregenomic RNA (pgRNA) by the viral reverse transcriptase (RT). The HBc protein is a small (183 amino acid residues long) protein that forms the shell of the NC and actively participates in multiple steps of HBV replication (4, 5). HBc contains an N-terminal domain (NTD) (from residue 1-140) and a C-terminal domain (CTD) (150-183), which are connected together by a short linker peptide (141-149). Whereas the NTD is essential for capsid assembly, it also plays a role in pgRNA packaging and reverse transcription. Similarly, the CTD is critical for pgRNA packaging and reverse transcription while also facilitates capsid assembly. Furthermore, the short linker also plays a role in multiple steps of HBV replication (6).

Use of HBc polypeptides comprising these mutations and combinations thereof are included in the disclosure. Use of cell cultures and cell lines comprising polynucleotides encoding the HBc mutant polypeptides, and non-human animals engineered to express the HBc mutant polypeptides either episomally or by integration into the genome, are also included in this disclosure, as described further below.

Test agents identified using compositions and methods of this disclosure will have relevance to human and non-human animals. In certain embodiments, compounds identified using the compositions and methods of will be useful for, among other purposes, inhibiting reproduction and/or growth of HBV, and/or for prophylaxis and/or therapy for disorders that are correlated with HBV infection. Thus, the present disclosure relates to a system to identify agents that can be used in pharmaceutical approaches to treating HBV infection that will be suitable for human and veterinary uses. In general, test agents comprises small molecules compounds, such as small molecule drugs or drug candidates, e.g., small molecule drugs or drug candidates that are tested in the described method to determine their effect on cccDNA formation. In embodiments, the small molecule generally has a low molecular weight, such as less than 900 Daltons. Representative and non-limiting examples of test agents and the characteristics of test agents are described in, for example, U.S. Pat. No. 9,657,013, from which the entire disclosure is incorporated herein by reference. In embodiments, a test agent from the disclosure of U.S. Pat. No. 9,657,013 may be used as a control to compare the effects of a test agent having an unknown function on cccDNA formation in the described method.

In general, this disclosure provides approaches which include but are not limited to making and using single-celled or multi-cellular eukaryotic organisms as a system to express and analyze the function of test agents by introducing into the organisms a polynucleotide encoding one or a combination of the HBc mutants described herein. In embodiments the polynucleotide comprises an expression vector. Any expression vector can be used and will be dependent upon the type of expression system (i.e., the type of cells) that are used and can be selected by one skilled in the art, given the benefit of the present disclosure. In embodiments, the expression vector comprises a polynucleotide that contains HBV DNA. Polynucleotides encoding the HBc mutants may be maintained as episomal elements, or they may be transiently or stably integrated into a chromosome of any particular eukaryotic cell. In embodiments, expression of the HBc mutants is constitutive, or is conditional, such as use of a cell-specific or tissue-specific promoter, or an inducible promoter to drive expression of the HBc mutants.

In certain embodiments, the HBc mutant is encoded on a single expression vector which does not encode all, or only encodes some, of the other HBV proteins (non HBc proteins) that are required for HBV expression. Such proteins are well known in the art. In other embodiments, the HBc mutant is encoded by the same expression vector that also encodes all of the other HBV components required for HBV expression. In embodiments, the HBV proteins are encoded by four open reading frames (ORFs) that are translated into viral core protein, surface proteins, polymerase/reverse transcriptase, and HBx. Thus, in embodiments the one or more vectors encode all or some of the mutant HBc, HBs, HBV polymerase, and HBx.

For ease of reference, cells engineered to express an HBc mutant according to the method of the invention are from time to time referred to herein as “HBc mutant+” cells.

In one embodiment, the method comprises providing a plurality of distinct samples comprising HBc mutant+ cells. In one embodiment, each sample expresses the same HBc mutant or mutant combination. In alternative embodiments, some or all of the samples express a different HBc mutant, or a different combination of mutants. The plurality of HBc mutant+ cell samples in this cell culture approach is configured so as to be amenable for high throughput screening (HTS). In certain embodiments, the samples are divided into a plurality of reaction chambers, such as wells in a plate. Any multi-well plate or other container can be used. In certain approaches, one or more 384-wells plates are used.

The method includes exposing each HBc mutant+ cell sample to at least one test agent, allowing a period of time for the test agent to be in contact with the HBc mutant+ cell samples, and subsequently determining HBV cccDNA, wherein a reduction in HBV cccDNA is indicative that the test agent is a candidate for use in prophylaxis and/or therapy of HBV infection. The same approach can be adapted for use with a plurality of modified animal models, as discussed above.

The amount of cccDNA can be assessed using any suitable approach. The current gold standard for detection of cccDNA is Southern blot analysis following resolution of cccDNA on an agarose gel, which clearly separates cccDNA from the other viral DNA species such as rcDNA. Additionally, the identity of cccDNA can be further verified by its resistance to heat denaturation and linearization following a single cut restriction digestion (see for example, Formation of hepatitis B virus covalently closed circular DNA: removal of genome-linked protein. J Virol 81:6164-6174)). For clinical/tissue samples that may contain few infected cells and thus very low levels of cccDNA an amplification process is preferred to detect the low levels of cccDNA. Relatively selective amplification strategies for sensitive detection of cccDNA via PCR or rolling circle amplification (RCA) have been developed and can be used in quantification of low levels of cccDNA normally found in tissue samples, as described further below.

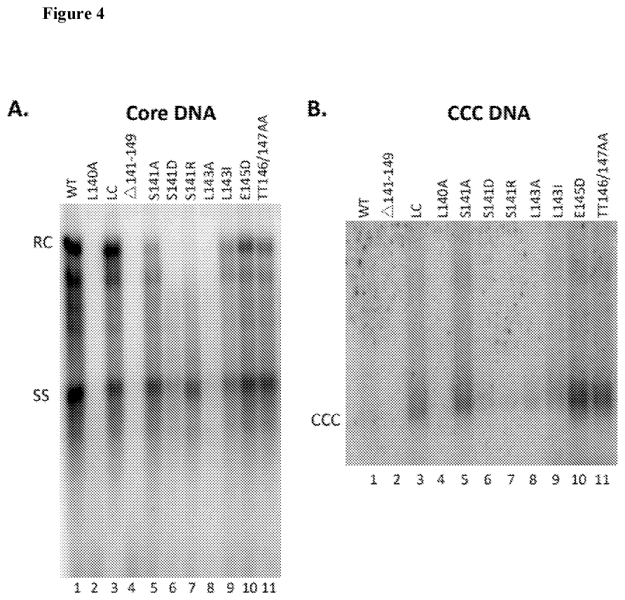

In certain embodiments, PCR-based approaches can be used, including but not necessarily limited to nested PCR reactions with suitable primers. In embodiments, primers spanning across the gap in the minus strand and primers corresponding to the variable region on the plus strand can be used to amplify non-interrupted cccDNA. In particular, quantitative measurement of HBV cccDNA may occur wherein in an HBV genome, the incomplete plus strand has a variable 3′ end but there is a defined 5′ end around position 1600 near direct repeat 2 (DR2), while the complete minus strand has defined 5′ and 3′ ends with a terminal redundancy of 9 bases. There is a gap near position 1800 near direct DR1. Therefore, primers can be designed and used to selectively amplify DNA fragments from replication intermediate cccDNA, but not from viral genome DNA, by taking advantage of sequence and structural differences between the viral genome and cccDNA. In one approach an RT-PCR technique is used, such as that described in U.S. Patent Publication No. 20040058314, from which the description of reagents and methods for detecting and quantifying HBV cccDNA is incorporated herein by reference. In certain approaches the disclosure involves use of RCA to selectively amplify circular DNA, which may then be further amplified and quantified. In some examples, a sample comprising the cccDNA can be processed using one or more nucleases to selectively digest rcDNA, replicative dsDNA and ssDNA. In embodiments, the so-called “Invader Assay” can be used (see, for example, Kwiatkowski R W, et al., Clinical, genetic and pharmacogenetic applications of the Invader assay. Mol. Diagn. 1999. 4:353-364). In embodiments a droplet digital polymerase chain reaction can be used (see, for example, Mu, D., et al. A sensitive and accurate quantification method for the detection of hepatitis B virus covalently closed circular DNA by the application of a droplet digital polymerase chain reaction amplification system. Biotechnology Letters. October 2015, Volume 37, Issue 10, pp 2063-2073). In embodiments, real-time PCR can be used (see, for example, Takkenberg, et al. Validation of a sensitive and specific real-time PCR for detection and quantitation of hepatitis B virus covalently closed circular DNA in plasma of chronic hepatitis B patients. J Med Virol. 2009 Jun;81(6):988-95.) In embodiments, cccDNA can be detected and amounts determined by electrophoretic techniques which allow separation and detection of cccDNA. (See, for example, Cai, et al., A Southern Blot Assay for Detection of Hepatitis B Virus Covalently Closed Circular DNA from Cell Cultures, Methods in Molecular Biology, Volume: 1030 (2013) pgs 151-161). In certain embodiments, cccDNA is detected as generally described herein for FIG. 4, and/or as described in Gao W, Hu J. 2007. Formation of hepatitis B virus covalently closed circular DNA: removal of genome-linked protein. J Virol 81:6164-6174, from which the description of obtaining and detecting cccDNA and differentiating cccDNA from other HBV polynucleotides is incorporated herein by reference.

For the animal models described herein, a biological sample can be obtained from the animal model and tested directly, or it can be subjected to one or more processing steps to, for example, isolate particular cell types or tissue types. In certain embodiments the biological sample is a liquid biological sample, such as blood, plasma, or urine. In other embodiments the sample comprises a biopsy. In certain approaches cccDNA can be detected in situ using at least in part tissue samples, such as tissue sections embedded in a substrate, such as a paraffin-embedded tissue. cccDNA can also be extracted from the tissue sections, or liver tissue biopsy or autopsy and quantified using any suitable approach.

In embodiments, the effect of a test agent on cccDNA in a cell culture or a sample from an animal model can be compared to a reference. Any suitable control can be used as a reference, including but not limited to a cell culture to which a test agent has not been added, or to which an agent with a known effect on cccDNA is added, or the reference can be a standardized reference, such as a known value that relates to HBV replication and/or cccDNA levels. In embodiments, the reference is a positive control, or a negative control. In embodiments, the reference is an amount of cccDNA that is produced by a control, i.e., a wild type HBc, or a different mutant HBc.

With respect to non-human mammals that are encompassed by the present disclosure as described above, it includes transient and non-transient introduction of a polynucleotide encoding an HBc mutant into cells, wherein the polynucleotide is maintained episomally, or is integrated into a chromosome of the mammal.

In certain approaches, polynucleotides can be introduced into neonatal or adult mice via injection into certain tissues such as the liver, using modified retroviral approaches, or by hydrodynamic gene delivery. Each of these approaches is well known in the art and can be readily adapted for use with the present disclosure. In certain embodiments the polynucleotides are introduced into the liver only, or are selectively expressed in the liver only, such as by being linked to a promoter that is only operative in hepatic cells.

In another aspect the disclosure comprises making and using transgenic non-human mammals, such as mice or pigs, wherein a polynucleotide encoding an HBc mutant is introduced into at least one chromosome of the non-human mammal. Compositions and methods for making homologous replacements of genes (i.e., gene knockins) in mice are well known in the art, and can be adapted for use with the present disclosure using any vector that is suitable for creating murine embryonic stem cells (ES). In embodiments, the polynucleotide can be integrated randomly into a chromosome of an ES cell, or a targeted insertion using a vector for recombination between homologous sequences can be used. As such, the vector is adapted to facilitate homologous recombination with a segment of murine chromosome. In general, a vector that is adapted for use in homologous combination with a target site in a recipient chromosome will have features known to those skilled in the art. These features include but are not necessarily limited to a selectable marker, such as the well characterized Neo marker, polycloning sites into which the HBc mutant gene segment is cloned as well as for inserting gene segments that are the same as those in the targeted portion of the chromosome so that the replacement is targeted correctly, a site for linearization of the vector, sites which are recognized by enzymes involved in certain recombination events, such as LoxP sites, and promoter sequences.

In another approach a polynucleotide encoding any of the HBc mutants (and additional HBV proteins if desired) can be integrated into the chromosome(s) of a non-human mammal. Methods of integrating polynucleotides into chromosomes are known in the art and include approaches such as homologous recombination of any polynucleotide of interest into chromosomes of, for example, embryonic stem cells, such that a transgenic mammal can be developed using established techniques as described further above. In certain embodiments, the disclosure includes a transgenic mammal wherein an HBc mutant polypeptide is expressed at least in the liver of the mammal. In embodiments, a CRISPR-based approach may be used. In embodiments, this aspect comprises introducing into any suitable cells, such as embryonic stem cells, or hepatic cells, or hepatic precursor cells, a CRISPR system that includes a suitable Cas enzyme, such as Cas9, but other Cas enzymes can be used, one or more suitable guide RNAs (gRNAs) and a DNA repair template. Generally, the CRISPR system includes a repair template that comprises a polynucleotide encoding the one or more HBc mutations such that the mutant HBc proteins are integrated by operation of the Cas enzyme and the guide RNA, and other CRISPR system components if necessary, which will be understood by those skilled in the art, into a desired location of the genome. The mutant HBc protein is accordingly produced endogenously by the modified non-human animal. In some embodiments, the non-human mammal is a mouse, but other non-human mammals can be used, including but not necessarily limited to non-human primates, canines, and porcine animals. Other viral components or polynucleotides may also be integrated into the non-human mammal genome, such that viral particles are produced. In this regard, the identification of HBV mutants with enhanced cccDNA formation as describe herein may facilitate the development of a non-human mammal, such as a mouse model, that is fully permissive for HBV infection. Such non-human constitute a significant breakthrough, not only for studying the fundamental mechanisms of HBV replication and pathogenesis, but also for developing more effective strategies for preventing or treating HBV infection. In particular, whereas mouse hepatocytes can support most steps of HBV replication, they fail to form HBV cccDNA and thus remain non-susceptible to HBV infection even after expression of the human cell receptor for the virus (11, 12). The failure of mouse hepatocytes to form cccDNA is likely related to the inability of the HBV capsids to undergo uncoating, which is, as described above, a prerequisite step in cccDNA formation by releasing the rcDNA for conversion to cccDNA (13, 14). Thus, capsid disassembly or uncoating represents a major determinant of HBV host species tropism. Overcoming the block to capsid disassembly, e.g., by construction of capsid mutants with enhanced uncoating capacity, is therefore included in this disclosure, and may be expected to overcome the block in mouse hepatocytes to cccDNA formation and render them fully permissive for HBV replication (15).

It will be apparent from the foregoing that the disclosure includes making any of several types of genetically modified non-human mammals that express a mutant HBc of this disclosure, and such genetically modified non-human mammals are encompassed by this disclosure for use with any method that involves HBV testing, analysis, and research. In certain non-limiting embodiments, the genetically modified mammal can be used to test candidate agents according to the method of the invention. In order to test candidate agents for use in anti-HBV applications, the test agents can be administered to such an animal, and a biological sample obtained from the mammal can be tested to determine if administering the agent reduced cccDNA. The test agent can be administered to modified mammal via any suitable route, including but not limited to parenteral, subcutaneous, intraperitoneal, intrapulmonary, and intranasal approaches. Parenteral infusions include intramuscular, intravenous, intra-arterial, intraperitoneal, pulmonary instillation as mist or nebulization, and subcutaneous administration. The test agent could also be introduced as a component of a food or drinking item. More than one animal can be used to test distinct agents, and in certain embodiments, more than one agent can be tested in any particular animal.

In embodiments a non-human mammal can be used in methods of this disclosure by infecting the animals with HBV that comprises one or more HBc core mutations that are described herein. In embodiments, polynucleotides encoding the HBV, or only the HBc, can be introduced into the non-human animal. In embodiments, a nucleoprotein comprising the HBc proteins and polynucleotides can be introduced into the non-human mammal. In embodiments, naked polynucleotides, or polynucleotides in a mixture of reagents to facilitate transfer of the polynucleotides into the nucleus can be used. In embodiments, such reagents include but are not limited to nanoparticles, liposomes, lipofectamine, and the like. The test agents can be administered in any form, including alone, or as a component of a composition of matter, such as a pharmaceutical formulation, and can be provided in liquid, solid, and semi-solid forms, tablets, capsules, granules, as a component of a feed, etc.

In certain embodiments, the disclosure includes comparing the effect of the one or more test agents to any suitable reference. The reference can be, for example, a control mammal that does not have HBV, or a mammal that does have HBV but is not given the test agent, or it can be a known value or range of values, or may be a value or range of values determined from analysis of a cohort of subjects. In embodiments, the reference comprises a statistical value, such as an area under a curve, or another area or plot on a graph, obtained from repeated measurements of cccDNA or a suitable alternative.

The disclosure includes fixing the results from analyzing test agents in a tangible medium, such as a computer file, and further comprises making a database of test results from analysis of a plurality of test agents. Such databases are also included in the scope of this disclosure. Thus, in embodiments, the disclosure includes a database or other compilation of results fixed in a tangible medium that comprises analysis of a plurality of test agents that can reduce HBV cccDNA, and an indication of test agents that are candidates for treating human HBV infection as determined using any method described herein. In embodiments, the database comprises results obtained from testing more than 10, more than 100, or more than a 1,000 test agents.

The following Example is provided to illustrate the invention, but are not intended to be limiting in any way.

### EXAMPLE 1

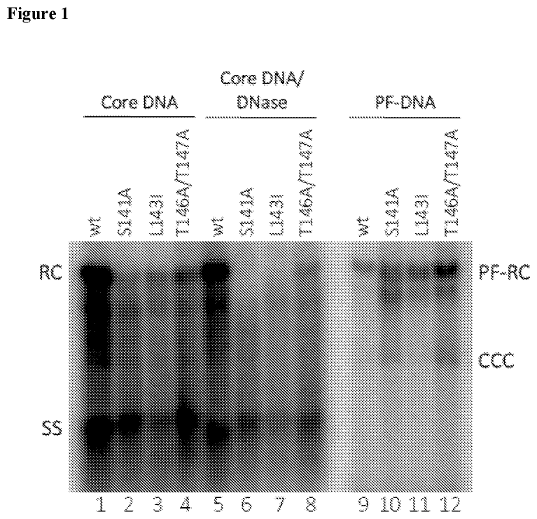

This Example describes substitution mutants within the HBc linker and their effects on viral replication in transfected human hepatoma cells. We found that three mutants, S141A, L143I, and T146A/T147A, showed enhanced cccDNA levels (by 10-30 fold compared to the WT HBc) (FIG. 1, lanes 9-12).

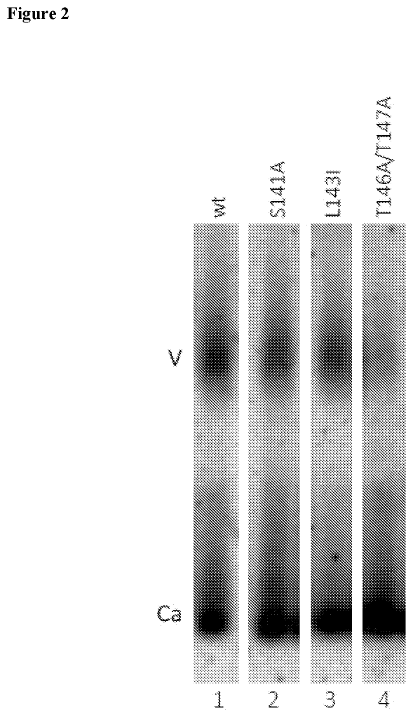

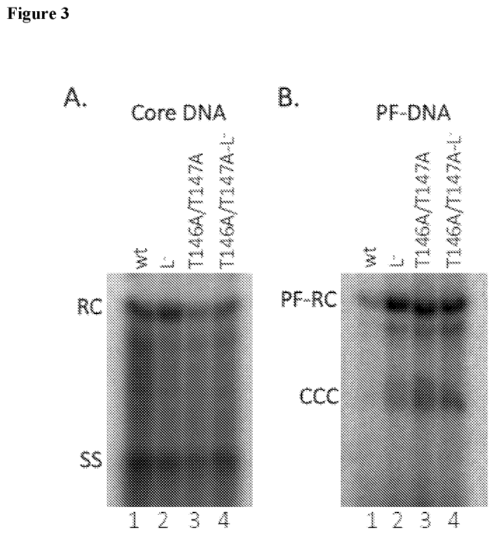

Without intending to be bound by any particular theory, it is considered that these HBc linker mutants increased cccDNA formation through one of two possible mechanisms. The S141A and L143I mutants showed hyper-destabilized NCs containing rcDNA (i.e., enhanced uncoating), as evidenced by the selective digestion of the rcDNA in the uncoating NCs by exogenous DNase (FIG. 1, lanes 6 & 7 vs. 2 & 3), which is similar to the previously identified I126A mutant (7). On the other hand, the T146A/T147A double substitution mutant increased cccDNA levels mainly by blocking the interactions of the NC containing rcDNA with the viral envelope proteins (thus blocking the secretion of complete virions) (FIG. 2, lane 4). T146A/T147A increased cccDNA to a level that was only slightly above that from an HBV mutant that retains the wildtype HBc but is unable to express the large envelope protein required for virion formation (FIG. 3, lanes 2 & 3). Combining the T146A/T147A and the envelope mutation only further enhanced cccDNA levels slightly above those from the envelope-defective mutant (FIG. 3, lane 4 vs. 2), which may be explained by the partially enhanced uncoating caused by T146A/T147A (FIG. 1, lane 8 vs. 4).

FIG. 4 demonstrates that HBc linker mutants increased HBV CCC DNA by 2-30 fold. These experiments were performed using the following mutations: (S141T/V148I/V149I, aka LC), S141A, S141D, S141R, L143A, L143I, E145D, TT146/147AA.

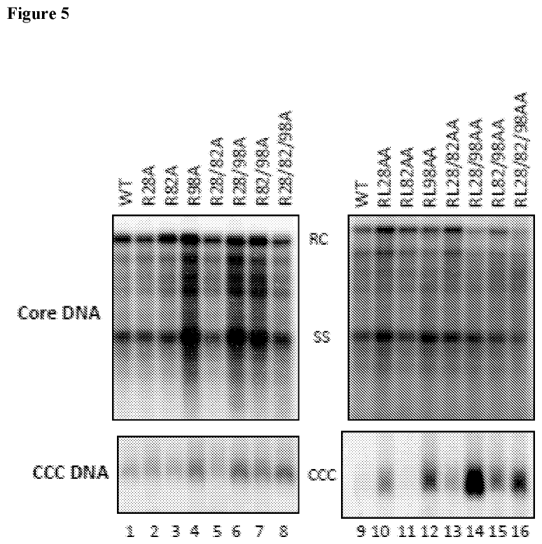

FIG. 5 demonstrated that HBc NTD mutants increased HBV CCC DNA by 2-40 fold. In particular, three RXL motifs in HBc NTD (R28, R82, R98) targeted, either R alone to A (R28A, R82A, R98A), or R and L in the motif both changed to A (RL28AA, RL82AA, RL98AA). Data are also shown for various combinations of mutations in two or three motifs: R28/82A, a combination of R28A and R82A; R28/98A, a combination of R28A and R98A; R82/98A, a combination of R82A and R98A; R28/82/98A, a combination of R28A, R82A, and R98A; RL28/82AA, a combination of R28A, L30A, R82A, and L84A; RL28/98AA, a combination of R28A, L30A, R98A, and L100A; RL82/98AA, a combination of R82A, L84A, R98A, and L100A; and RL28/82/98AA, a combination of R28A, L30A, R82A, L84A, R98A, and L100A.

The following reference listing is not an indication that any particular reference is material to patentability.
1. Hu J. 2016. Hepatitis B virus virology and replication, p 1-34. In Liaw Y-F, Zoulim F (ed), Hepatitis B virus in human diseases. Humana Press, Springer Cham Heidelberg New York Dordrecht London.
2. Hu J, Protzer U, Siddiqui A. 2019. Revisiting Hepatitis B virus: Challenges of Curative Therapies. J Virol doi:10.1128/JVI.01032-19.
3. Hu J, Seeger C. 2015. Hepadnavirus Genome Replication and Persistence. Cold Spring Harb Perspect Med 5:a021386.
4. Ludgate L, Liu K, Luckenbaugh L, Streck N, Eng S, Voitenleitner C, Delaney WEt, Hu J. 2016. Cell-Free Hepatitis B Virus Capsid Assembly Dependent on the Core Protein C-Terminal Domain and Regulated by Phosphorylation. J Virol 90:5830-44.
5. Zlotnick A, Venkatakrishnan B, Tan Z, Lewellyn E, Turner W, Francis S. 2015. Core protein: A pleiotropic keystone in the HBV lifecycle. Antiviral Res 121:82-93.
6. Liu K, Luckenbaugh L, Ning X, Xi J, Hu J. 2018. Multiple roles of core protein linker in hepatitis B virus replication. PLoS Pathog 14:e1007085.
7. Cui X, Luckenbaugh L, Bruss V, Hu J. 2015. Alteration of Mature Nucleocapsid and Enhancement of Covalently Closed Circular DNA Formation by Hepatitis B Virus Core Mutants Defective in Complete-Virion Formation. J Virol 89:10064-72.
8. Berke J M, Dehertogh P, Vergauwen K, Van Damme E, Mostmans W, Vandyck K, Pauwels F. 2017. Capsid Assembly Modulators Have a Dual Mechanism of Action in Primary Human Hepatocytes Infected with Hepatitis B Virus. Antimicrob Agents Chemother 61:e00560-17.
9. Guo F, Zhao Q, Sheraz M, Cheng J, Qi Y, Su Q, Cuconati A, Wei L, Du Y, Li W, Chang J, Guo J T. 2017. HBV core protein allosteric modulators differentially alter cccDNA biosynthesis from de novo infection and intracellular amplification pathways. PLoS Pathog 13:e1006658.
10. Lahlali T, Berke J M, Vergauwen K, Foca A, Vandyck K, Pauwels F, Zoulim F, Durantel D. 2018. Novel Potent Capsid Assembly Modulators Regulate Multiple Steps of the Hepatitis B Virus Life Cycle. Antimicrob Agents Chemother 62.
11. Guidotti L G, Matzke B, Schaller H, Chisari F V. 1995. High-level hepatitis B virus replication in transgenic mice. Journal of Virology 69:6158-69.
12. He W, Ren B, Mao F, Jing Z, Li Y, Liu Y, Peng B, Yan H, Qi Y, Sun Y, Guo J T, Sui J, Wang F, Li W. 2015. Hepatitis D Virus Infection of Mice Expressing Human Sodium Taurocholate Co-transporting Polypeptide. PLoS Pathog 11:e1004840.
13. Cui X, Guo J T, Hu J. 2015. Hepatitis B Virus Covalently Closed Circular DNA Formation in Immortalized Mouse Hepatocytes Associated with Nucleocapsid Destabilization. J Virol 89:9021-8.
14. Lempp F A, Qu B, Wang Y X, Urban S. 2016. Hepatitis B Virus Infection of a Mouse Hepatic Cell Line Reconstituted with Human Sodium Taurocholate Cotransporting Polypeptide. J Virol 90:4827-31.
15. Hu J, Lin Y Y, Chen P J, Watashi K, Wakita T. 2019. Cell and Animal Models for Studying Hepatitis B Virus Infection and Drug Development. Gastroenterology 156:338-354.

While the invention has been described through specific embodiments, routine modifications will be apparent to those skilled in the art and such modifications are intended to be within the scope of the present invention.

1. A method for identifying a test agent for use in reducing Hepatitis B virus (HBV) in an individual, the method comprising:
a) introducing at least one test agent into a mammalian cell culture comprising mammalian cells, the cells comprising a mutated HBV core (HBc), wherein the mutated core comprises at least one HBc mutation in SEQ ID NO:1 that is at position 28, 30, 82, 84, 98, 100, 141, 143, 145, 146, 147, 148 or 149 of SEQ ID NO:1;
b) allowing the test agent to be in contact with the mammalian cells for a period of time, and subsequently,
c) determining amounts of HBV covalently closed circular (ccc) DNA in the mammalian cells, wherein a reduction in cccDNA relative to a control indicates the at least one test agent is a candidate for reducing HBV in an individual.

2. The method of claim 1, wherein the mutation is a mutation of SEQ ID NO:1 that is at least one of R28A, L30A, R82A, L84A, R98A, L100A, S141A, S141D, S141R, S141T, L143A, L143I, E145D, T146A, T147A, V148I, and V149I.

3. The method of claim 2, wherein the at least one HBc mutation comprises a combination of the said mutations.

4. The method of claim 3, wherein the combination of mutations comprises one of R28A/R82A, R28A/R98A, R82A/R98A, R28A/R82A/R98A, R28A/L30A, R82A/L84A, R98A/L100A, T146A/T147A, and S141T/V148I/V149I.

5. The method of claim 1, wherein the control comprises a cccDNA value produced by a control mammalian cell culture comprising an HBc that does not comprise the at least one HBc mutation in SEQ ID NO:1.

6. The method of claim 5, wherein the mammalian cell culture is separated into a plurality of separate test containers, and wherein a distinct test agent is added into each test container.

7. The method of claim 6, wherein the plurality of test containers are comprised by a multi-well plate.

8. A method for screening test agents comprising:
a) introducing one or more test agents into a non-human mammalian subject in which cells in the non-human mammalian subject contain a mutated HBV core (HBc) protein, wherein the mutated HBc protein comprises at least one mutation that is at position 28, 30, 82, 84, 98, 100, 141, 143, 145, 146, 147, 148 or 149 of SEQ ID NO:1; and
b) determining amounts of HBV cccDNA in the mammal, wherein a reduction in the cccDNA relative to a control indicates the test agent is a candidate for use in reducing HBV in an individual.

9. The method of claim 8, wherein the at least one HBc mutation a combination of the mutations.

10. The method of claim 9, wherein the combination of mutations comprises one of R28A/R82A, R28A/R98A, R82A/R98A, R28A/R82A/R98A, R28A/L30A, R82A/L84A, R98A/L100A, T146A/T147A, and S141T/V148I/V149I in SEQ ID NO:1.

11. The method of claim 1, wherein the control comprises a cccDNA value obtained from a non-human mammal comprising a HBc protein comprising an HBc that does not comprise the at least one HBc mutation in SEQ ID NO:1.

12. A polynucleotide encoding at least one HBc mutation comprising an amino acid change at HBc protein position 28, 30, 82, 84, 98, 100, 143, 145, 146, 147, 148, or 149 of SEQ ID NO:1.

13. The polynucleotide of claim 12 encoding a combination of the amino acid changes.

14. The polynucleotide of claim 13, wherein the combination of amino acid changes is selected from R28A/R82A, R28A/R98A, R82A/R98A, R28A/R82A/R98A, R28A/L30A, R82A/L84A, R98A/L100A, T146A/T147A, and S141T/V148I/V149I in SEQ ID NO:1.

15. An expression vector comprising the polynucleotide of claim 12.

16. A mammalian cell comprising the polynucleotide of claim 12.

17. A non-human transgenic animal comprising the polynucleotide of claim 12.

18. The non-human transgenic animal comprising the polynucleotide of claim 17, wherein the at least one mutation of SEQ ID NO:1 is at least one of R28A, L30A, R82A, L84A, R98A, L100A, S141A, S141D, S141R, S141T, L143A, L143I, E145D, T146A, T147A, V148I, and V149I.

19. Isolated Hepatitis B virus (HBV) viral particles obtained from mammalian cells into which the polynucleotide of claim 12 has been introduced.

20. The isolated HBV particles of claim 19, wherein the mutation is a mutation of SEQ ID NO:1 that is at least one of R28A, L30A, R82A, L84A, R98A, L100A, S141A, S141D, S141R, S141T, L143A, L143I, E145D, T146A, T147A, V148I, and V149I.
